(19) United States
(12) Patent Application Publication
(10) Pub. No.: US 2006/0184236 A1
(43) Pub. Date: Aug. 17, 2006

(54) Intraluminal device including an optimal drug release profile, and method of manufacturing the same

(75) Inventors: Ryan Alexander Jones, Higley, AZ (US); Dave Doty, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/057,094

(22) Filed: Feb. 11, 2005

(51) Int. Cl. A61F 2/06

(52) U.S. Cl. 623/1.42

(57) ABSTRACT

An intraluminal device, an intraluminal stent delivery system, and a method of manufacturing the same. The device includes a body. At least one therapeutic agent coating is positioned on the body and positioned substantially on an interface portion to provide an optimal drug release profile. The stent delivery system includes a catheter and a stent disposed on the catheter. The stent comprises a body including at least one therapeutic agent coating positioned on the body and positioned substantially on an interface portion to provide an optimal drug release profile. The manufacturing method includes providing a body and applying at least one therapeutic agent coating positioned on the body and distributing the at least one therapeutic agent coating substantially on an interface portion to provide an optimal drug release profile.

### TECHNICAL FIELD OF THE INVENTION

The present invention relates generally to the field of implantable medical devices. More particularly, the invention relates to an intraluminal device including an optimal drug release profile, and a method of manufacturing the same.

### BACKGROUND OF THE INVENTION

Balloon angioplasty has been used for the treatment of narrowed and occluded blood vessels. A frequent complication associated with the procedure is restenosis, or vessel re-narrowing. Within 3-6 months of angioplasty, an unacceptably high degree and incidence of restenosis occurs. To reduce the incidence of re-narrowing, several strategies have been developed. Implantable devices, such as stents, have been used to reduce the rate of angioplasty related restenosis by about half. The use of such intraluminal devices has greatly improved the prognosis of these patients. Nevertheless, restenosis remains a formidable problem associated with the treatment of narrowed blood vessels.

Restenosis associated with interventional procedures such as balloon angioplasty may occur by at least two mechanisms: thrombosis and intimal hyperplasia. During angioplasty, a balloon is inflated within an affected vessel thereby compressing the blockage and imparting a significant force, and subsequent trauma, upon the vessel wall. The natural antithrombogenic lining of the vessel lumen may become damaged thereby exposing thrombogenic cellular components, such as matrix proteins. The cellular components, along with the generally antithrombogenic nature of any implanted materials (e.g., a stent), may lead to the formation of a thrombus, or blood clot. The risk of thrombosis is generally greatest immediately after the angioplasty.

A second mechanism of restenosis is intimal hyperplasia, or excessive tissue re-growth. The trauma imparted upon the vessel wall from the angioplasty is generally believed to be an important factor contributing to hyperplasia. This exuberant cellular growth may lead to vessel “scarring” and significant restenosis. The risk of hyperplasia associated restenosis is usually greatest 3 to 6 months after the procedure.

Prosthetic devices, such as stents, may be implanted during interventional procedures such as balloon angioplasty to reduce the incidence of vessel restenosis. To improve device effectiveness, stents may be coated with one or more therapeutic agents providing a mode of localized drug delivery. The therapeutic agents are typically intended to limit or prevent the aforementioned mechanisms of restenosis. For example, antithrombogenic agents such as heparin or clotting cascade IIb/IIIa inhibitors (e.g., abciximab and eptifibatide) may be coated on the stent thereby diminishing thrombus formation. Such agents may effectively limit clot formation at or near the implanted device. Some antithrombogenic agents, however, may not be effective against intimal hyperplasia. Therefore, the stent may also be coated with antiproliferative agents or other agents to reduce excessive endothelial re-growth. Therapeutic agents provided as coating layer(s) on implantable medical devices may effectively limit restenosis and reduce the need for repeated treatments.

Several strategies have been developed for coating one or more therapeutic agents onto the surface of medical devices, such as stents. Standard methods may include dip coating, spray coating, and chemical bonding. The coating may be applied as a mixture, solution, or suspension of polymeric material and/or therapeutic agent dispersed in an organic vehicle or a solution or partial solution. Further, the coating may include one or more sequentially applied, relatively thin layers deposited in a relatively rapid sequence. The stent is typically in a radially expanded state during the application process. In some applications, the coating may include a composite initial primer coat, or undercoat, and a composite topcoat, or cap coat. The coating thickness ratio of the topcoat to the undercoat may vary with the desired effect and/or the elution system.

Current coating methodologies of medical devices typically provide a roughly uniform coating of the therapeutic agent(s) on its various surfaces. This uniform distribution may not provide optimal delivery of the agents to the tissue in which it is implanted (e.g., vascular endothelium). For example, much of the coating may be positioned on a portion of the medical device that does not contact the vessel wall and therefore is not effectively delivered. As such, it would be desirable to provide a strategy for coating a medical device that would provide optimal delivery of therapeutic agent(s) to the portion that interfaces the vessel wall in which it is implanted.

Accordingly, it would be desirable to provide an intraluminal device including an optimal drug release profile, and a method of manufacturing the same that would overcome the aforementioned and other limitations.

### SUMMARY OF THE INVENTION

A first aspect according to the invention provides an intraluminal device. The device includes a body. At least one therapeutic agent coating is positioned on the body and positioned substantially on an interface portion to provide an optimal drug release profile.

A second aspect according to the invention provides a stent delivery system. The system includes a catheter and a stent disposed on the catheter. The stent comprises a body including at least one therapeutic agent coating positioned on the body and positioned substantially on an interface portion to provide an optimal drug release profile.

A third aspect according to the invention provides a method of manufacturing an intraluminal device. The manufacturing method includes providing a body and applying at least one therapeutic agent coating positioned on the body and distributing the at least one therapeutic agent coating substantially on an interface portion to provide an optimal drug release profile.

The foregoing and other features and advantages of the invention will become further apparent from the following description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of an intraluminal stent delivery system including a stent mounted on a balloon and positioned within a sheath, in accordance with one embodiment of the present invention;

FIG. 2 is a detailed view of the stent of FIG. 1;

FIGS. 3A, 3B, and 3C are cross-sectional views of various strut geometries including a therapeutic agent coating layered thereon and positioned adjacent a vessel wall, in accordance with one embodiment of the present invention; and

FIG. 4 is a cross-sectional view of immobile and mobile polymeric layers positioned on a stent, in accordance with one embodiment of the present invention.

### DESCRIPTION OF THE PRESENTLY PREFERRED EMBODIMENTS

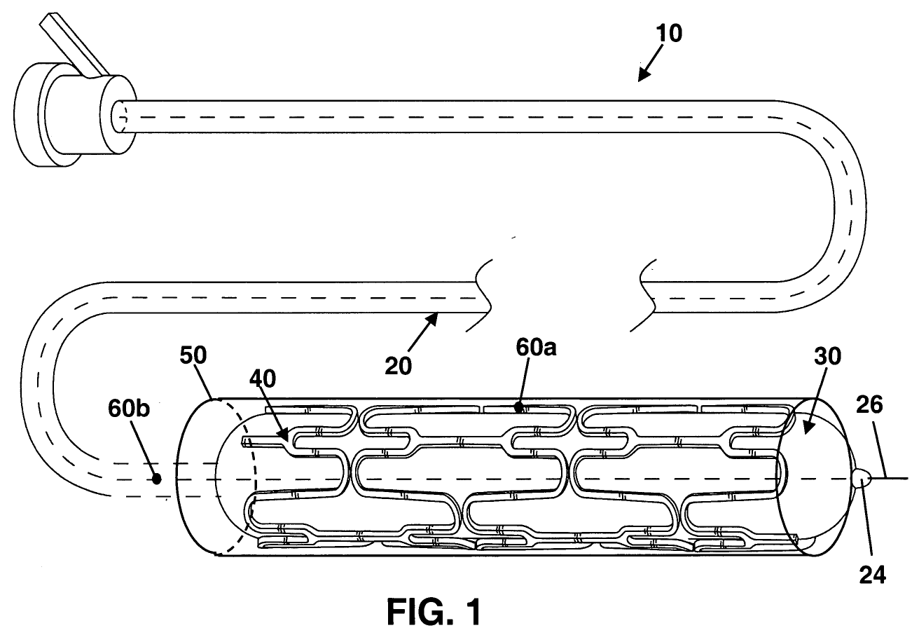

Referring to the drawings, which are not necessarily drawn to scale and wherein like reference numerals refer to like elements, FIG. 1 is a perspective view of an intraluminal stent delivery system in accordance with one embodiment of the present invention and shown generally by numeral 10. System 10 may include a catheter 20, a balloon 30 operably attached to the catheter 20, and a stent 40 disposed on the balloon 30. Balloon 30 may be any variety of balloons capable of expanding the stent 40. Balloon 30 may be manufactured from any sufficiently elastic material such as polyethylene, polyethylene terephthalate (PET), nylon, or the like. Stent 40 may be expanded in sympathy with the balloon 30. System 10 may include a sheath 50 to retain the stent 40 in a collapsed state and to prevent contact with surfaces, such as a vessel wall, during advancement through a vessel lumen and subsequent deployment. Once the stent 40 is properly positioned, the sheath 50 may be retracted thereby allowing the stent to assume its expanded shape. The advancement, positioning, and deployment of stents and similar devices are well known in the art.

The terms “catheter” and “stent”, as used herein, may include any number of intravascular and/or implantable prosthetic devices (e.g., a stent-graft); the examples provided herein are not intended to represent the entire myriad of devices that may be adapted for use with the present invention. Although the devices are described herein are primarily done so in the context of deployment within a blood vessel, it should be appreciated that intravascular and/or implantable prosthetic devices in accordance with the present invention may be deployed in other vessels, such as a bile duct, intestinal tract, esophagus, airway, etc.

Catheter 20 comprises an elongated tubular member manufactured from one or more polymeric materials, sometimes in combination with metallic reinforcement. In some applications (such as smaller, more tortuous arteries), it is desirable to construct the catheter from very flexible materials to facilitate advancement into intricate access locations. Numerous over-the-wire, rapid-exchange, and other catheter designs are known and may be adapted for use with the present invention. Catheter 20 may be secured at its proximal end to a suitable Luer fitting 22, and may include a distal rounded end 24 to reduce harmful contact with a vessel. Catheter 20 may be manufactured from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax®, Vestamid®, Tecoflex®, Halar®, Hyflon®, Pellathane®, combinations thereof, and the like. Catheter 20 may include an aperture formed at the distal rounded end 24 allowing advancement over a guidewire 26.

In another or the same embodiment, the catheter 20 may further include a drug delivery element for delivering drugs to the vessel during stent 40 deployment. The drug delivery element may include at least one elongated lumen formed within the catheter 20. As such, additional drugs may be administered to the patient during the deployment procedure.

Stent 40, particularly those of the self-expanding variety, may be positioned within the sheath 50 to retain the stent 40 in the collapsed state until it is at the deployment site. The sheath 50 may then be retracted thereby allowing the self-expanding stent 40 to assume its naturally expanded shape. The sheath 50 may also function to prevent the stent from inadvertent contact with other surfaces to, for example, prevent injury to a vessel wall or to maintain the integrity of a therapeutic agent coating of the stent 40. Self-expanding stents typically do not require a balloon to provide the radial forces needed to expand the stent. However, the balloon may provide other advantages such as ensuring proper placement of the stent within the vessel (i.e., to prevent the stent from slipping or ‘jumping’ due to its inherent spring-like properties).

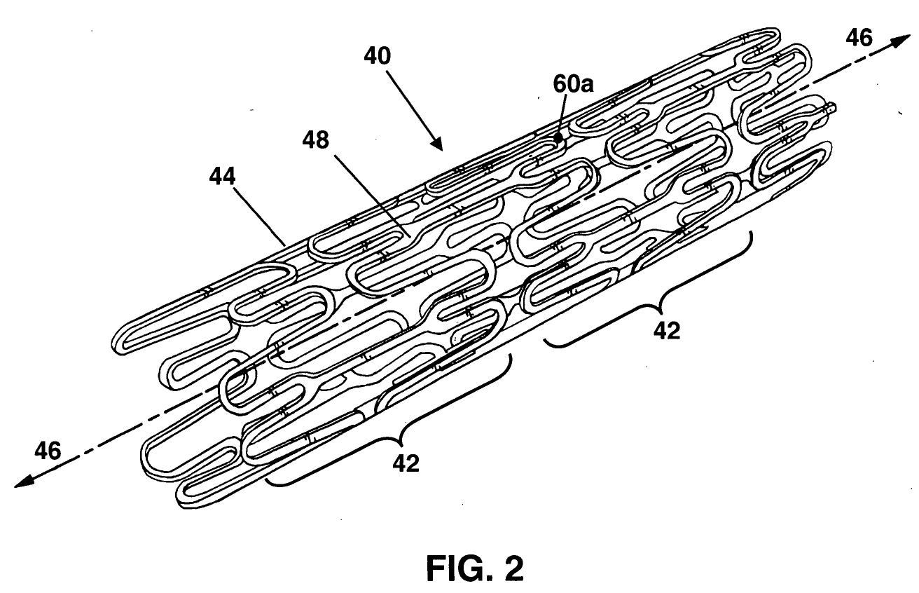

FIG. 2 is a detailed view of the stent 40 shown in FIG. 1. Stent 40 may be of any variety of implantable prosthetic devices as known in the art. Stent 40 may be manufactured from a skeletal framework or mesh of material forming a tube-like structure and may be capable of self-expanding or being expanded by another device such as a balloon or other means. In one embodiment, the stent 40 may include a plurality of identical cylindrical segments 42 arranged end-to-end. Those skilled in the art will recognize that the number of stent segments may vary and that numerous other stents, stent-grafts, and implantable prosthetic devices may be adapted for use with the present invention; the described stent 40 is provided merely as an example. For example, a stent-graft device for treatment of abdominal aortic aneurisms or other implantable prosthetic device may be adapted for use with the present invention.

In one embodiment, the stent 40 may include a generally tubular body 44 defining a passageway extending along a longitudinal axis 46. Stent 40 may include the plurality of cylindrical segments 42 arranged successively along the longitudinal axis 46. Each of the cylindrical segments 42 may have a length along the longitudinal axis 46 and may be comprised of at least one strut 48, which in this case are generally W-shaped. Struts 48 may open in alternating directions along the longitudinal axis 46 about the perimeter or circumference of the cylindrical segments 42. Stent 40 preferably is compressed into a smaller diameter (i.e., when “loaded” on the balloon and/or within the sheath 50) for deployment within a vessel lumen at which point the stent 40 may be expanded to provide support to the vessel. Once properly positioned within a vessel lumen, the sheath 50 is retracted as the balloon 30 and stent 40 expand. Cylindrical segments 42 may move radially outward from the longitudinal axis 46 as the stent 40 expands. At least one (radiopaque) marker 60a may be disposed on the stent 40, catheter 20, and or component thereof to allow in situ visualization and proper advancement, positioning, and deployment of the stent 40. The marker(s) may be manufactured from a number of materials used in the art for visualization including radiopaque materials such as platinum, gold, tungsten, metal, metal alloy, and the like. Marker(s) may be visualized by fluoroscopy, IVUS, and other methods known in the art. Those skilled in the art will recognize that numerous devices and methodologies may be utilized for deploying a stent and other intraluminal device in accordance with the present invention.

In one embodiment, the stent 40 may be expanded by the balloon 30 or some other means of providing outward radial forces. Stent 40 may be manufactured from an inert, biocompatible material with high corrosion resistance. The biocompatible material should ideally be plastically deformed at low-moderate stress levels. In another embodiment, the stent 40 may be manufactured from, for example, a nickel titanium alloy and/or other alloy(s) that exhibit superlastic behavior (i.e., capable of significant distortion without plastic deformation) thereby providing self-expanding properties. Suitable materials for stents include, but are not limited to, tantalum, stainless steel, titanium ASTM F63-83 Grade 1, niobium, high carat gold K 19-22, and MP35N. Furthermore, the stent material may include any number of other metallic and/or polymeric biocompatible materials recognized in the art for such devices.

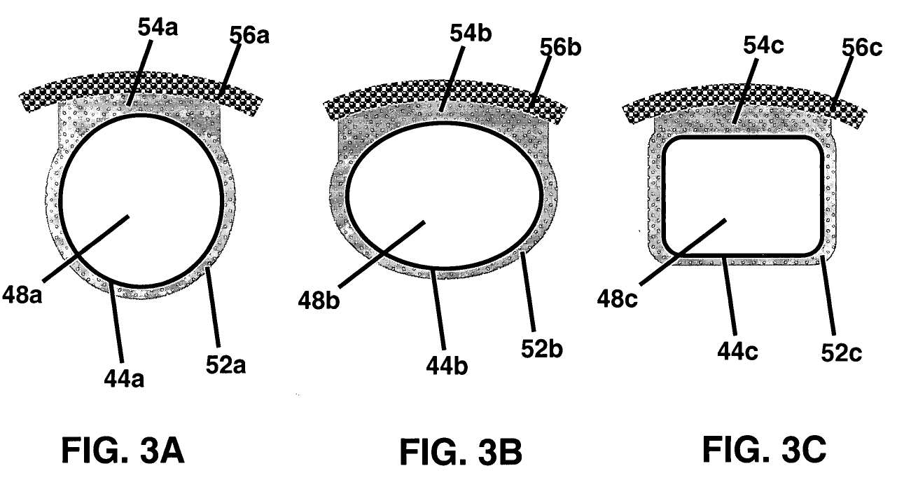

As shown in cross-sectional views FIGS. 3A, 3B, and 3C, three different strut 48a, 48b, and 48c geometries are illustrated. Bodies 44a, 44b, and 44c include at least one therapeutic agent coating 52a, 52b, and 52c positioned thereon. The at least one therapeutic agent coating 52a, 52b, and 52c is positioned substantially on an interface portion 54a, 54b, and 54c to provide an optimal drug release profile. Bodies 44a, 44b, and 44c are shown positioned adjacent a vessel wall 56a, 56b, and 56c. The interface portion 54a, 54b, and 54c is defined herein as a portion of the intraluminal device (e.g., the stent bodies 44a, 44b, and 44c including the at least one therapeutic agent coating 52a, 52b, and 52c) that is positioned adjacent the vessel wall 56a, 56b, and 56c when deployed. The optimal drug release profile is obtained by providing a substantial distribution of the at least one therapeutic agent coating (i.e., that part including one or more drugs), positioned at the interface portion 54a, 54b, and 54c. In one embodiment, the substantial distribution comprises a thicker layering of the at least one therapeutic agent coating at the interface portion 54a, 54b, and 54c. In another or the same embodiment, the substantial distribution comprises a greater concentration of the drug(s) at the interface portion 54a, 54b, and 54c. In another or the same embodiment, the substantial distribution comprises an optimized elution of the drug(s) at the interface portion 54a, 54b, and 54c. The profile of the shape of the at least one therapeutic agent coating 52a, 52b, and 52c at the interface portion 54a, 54b, and 54c may vary, but is preferably contoured to correspond to the vessel wall 56a, 56b, and 56c (e.g., a rounded, smooth shape) thereby minimizing potential trauma during contact.

In one embodiment, the interface portion 54a, 54b, and 54c may comprise an outer surface of the bodies 44a, 44b, and 44c. In another embodiment, the interface portion may comprise another portion or surface in addition to or in lieu of the outer surface of the intraluminal device body. This may be desirable, for example, for intraluminal devices including portions other than or in addition to the outer surface of the body that contact the vessel wall.

In many instances, when the intraluminal device (e.g., the stent) is deployed, the endothelium is injured most where the device contacts the vessel wall 56a, 56b, and 56c. As such, providing a substantial distribution of the at least one therapeutic agent coating positioned at the interface portion provides a more efficient use of the therapeutic agent coating(s) and an optimal drug release profile. Those skilled in the art will appreciate that the strut 48a, 48b, and 48c, the body 44a, 44b, and 44c, and positioning of the at least one therapeutic agent coating 52a, 52b, and 52c may vary from that illustrated and described herein.

In one embodiment, the therapeutic agent coating may comprise one or more drugs, polymers, solvents, a component thereof, a combination thereof, and the like. For example, the therapeutic agent coating may include a mixture of a drug and a polymer dissolved in a compatible liquid solvent as known in the art. Some exemplary drug classes that may be included are antiangiogenesis agents, antiendothelin agents, anti-inflammatory agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, and the like. Specific examples of drugs include abciximab, angiopeptin, colchicine, eptifibatide, heparin, hirudin, lovastatin, methotrexate, rapamycin, streptokinase, taxol, ticlopidine, tissue plasminogen activator, trapidil, urokinase, and growth factors VEGF, TGF-beta, IGF, PDGF, and FGF.

The polymer generally provides a matrix for incorporating the drug within the coating, or may provide means for slowing the elution of an underlying or incorporated therapeutic agent. Some exemplary biodegradable polymers that may be adapted for use with the present invention include, but are not limited to, polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymers blends, combinations thereof, and the like. Exemplary non-biodegradable polymers that may be adapted for use with the present invention may be divided into at least two classes. The first class includes hydrophobic polymers such as polyolefins, acrylate polymers, vinyl polymers, styrene polymers, polyurethanes, polyesters, epoxy, nature polymers, their copolymers, blends, and copolymer blends, combinations thereof, and the like. The second class includes hydrophilic polymers, or hydrogels, such as polyacrylic acid, polyvinyl alcohol, poly(N-vinylpyrrolidone), poly(hydroxy-alkylmethacrylate), polyethylene oxide, their copolymers, blends and copolymer blends, combinations of the above, and the like.

Solvents are typically used to dissolve the therapeutic agents comprising the coating. Some exemplary solvents that may be adapted for use with the present invention include, but are not limited to, acetone, ethyl acetate, tetrahydrofuran (THF), chloroform, N-methylpyrrolidone (NMP), and the like.

Those skilled in the art will recognize that the nature of the drugs, polymers, and solvent may vary greatly and are typically formulated to achieve a given therapeutic effect, such as limiting restenosis, thrombus formation, hyperplasia, etc. Once formulated, a therapeutic agent (mixture) comprising the coating(s) may be applied to the stent by any of numerous strategies known in the art including, but not limited to, spraying, dipping, rolling, nozzle injection, and the like. It will be recognized that the at least one therapeutic agent coating may be alternatively layered, arranged, configured on/within the stent depending on the desired effect. Before application, one or more primers may be applied to the stent to facilitate adhesion of the at least one therapeutic agent coating. Once the at least one therapeutic agent coating is/are applied, it/they may be dried (i.e., by allowing the solvent to evaporate) and, optionally, other coating(s) (e.g., a “cap” coat) added thereon. Numerous strategies of applying the primer(s), therapeutic agent coating(s), and cap coat(s) in accordance with the present invention are known in the art. The inventors contemplate numerous strategies for applying the at least one therapeutic agent coating as would be appreciated by one skilled in the art. In one embodiment, the at least one therapeutic agent coating and any additional layer(s) (e.g., primer, cap coat, polymeric layers) may be applied simultaneously or separately by, for example, differentially masking the stent. In another embodiment, the stent may be placed into a mold. The at least one therapeutic agent coating may be sprayed and/or injected into apertures formed in the mold and then cured via heat, dehydration, pressure (positive and negative), cross-linking (i.e., via ultraviolet light, chemicals, etc.), and the like. The process may be repeated to apply numerous coatings.

Polymeric layers known in the art may be utilized within or adjacent to the coatings to provide differential elution kinetics of the drug(s), particularly at the interface portion between the intraluminal device and the vessel wall. Polymeric layers include both soft, mobile layers as well as hard, immobile layers. The different nature of these polymeric layers, or in some cases, barriers, influences the dynamics of drug elution kinetics therethrough. As such, different orders of elution kinetics may be provided singularly or in combination on the intraluminal device. For example, a zero-order elution kinetic provides a drug elution rate that is relatively constant over time. This provides a steady, long lasting drug delivery. A first-order elution kinetic provides a drug elution rate that diminishes over time. This may be beneficial for treating more acute conditions, such as inflammation/trauma associated with the stenting procedure (i.e., by providing a “burst” of drug delivery after stent deployment). An asymmetric elution kinetic provides an increase of drug delivery after disruption of an outer polymeric layer. This may be beneficial to treat long-term conditions such as restenosis.

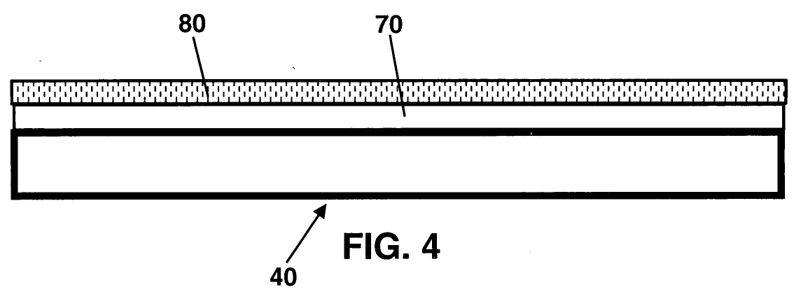

In one embodiment, as shown in the cross-sectional view of FIG. 4, an immobile polymeric layer 70 may be positioned on the stent 40 and may include one or more anti-inflammatory agent(s) dispersed therein. A mobile polymeric layer 72 may be positioned on top of the immobile polymeric layer 70 and may include an antiproliferative agent(s) dispersed therein.

In other embodiments, the layering and positioning of one or more of the mobile and immobile polymeric layers may differ. The type and number of the drug(s) included in the polymeric layers may also differ. The same drug(s) may also be released from each different polymeric layer. Preferably, the polymeric barriers, the therapeutic agent coating(s), and any primer(s)/cap coat(s) are layered substantially evenly on the stent providing a smooth surface that minimizes irritation to the vessel wall. Those skilled in the art will recognize that a myriad of polymeric layerings, configurations, and constitutions and any drug(s) included therein, and primer(s)/cap coat(s) may be provided in accordance with the present invention. This typically depends on the nature of the intraluminal device and the required release profile(s) of the drug(s).

The nature of the polymeric layer(s) may provide the differential elution kinetics. For example, a mobile polymeric layer may be positioned on top of the immobile layer including one or more drug(s). The mobile polymeric layer may provide a physical barrier slowing the elution of the drug(s) thereby providing a first-order elution kinetic. In addition, a similar layering configuration may provide an asymmetric elution kinetic. This configuration may be appropriate for situations requiring prevention of restenosis. The asymmetric elution kinetic occurs as a result of a delayed disruption of the top polymeric layer. Specifically, any subsequent restenosis of the vessel may physically disrupt the top polymeric layer which in turn may allow additional drug(s) to be released from the bottom polymeric layer. In effect, a second deployment procedure may not be required as additional drug(s) are released from the original intraluminal device by some stimulus such as mechanical disruption of the top polymeric layer.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

While the embodiments of the invention disclosed herein are presently considered to be preferred, various changes and modifications can be made without departing from the spirit and scope of the invention. For example, the intraluminal device is not limited to any particular design, such as a stent. In addition, the therapeutic agent coating composition, coating process, and positioning may be varied while providing an optimal drug release profile.

1. An intraluminal device comprising:
a body;
at least one therapeutic agent coating positioned on the body and positioned substantially on an interface portion to provide an optimal drug release profile.

2. The device of claim 1 wherein the body comprises a stent.

3. The device of claim 1 wherein the interface portion comprises an outer surface of the body.

4. The device of claim 1 wherein the at least one therapeutic agent coating comprises at least one drug.

5. The device of claim 4 wherein the at least one drug comprises at least one of an anti-inflammatory agent and an antiproliferative agent.

6. The device of claim 1 wherein the at least one therapeutic agent coating comprises at least one of an immobile polymeric layer coating and a mobile polymeric layer coating.

7. The device of claim 1 wherein the at least one therapeutic agent coating comprises a differential elution kinetic.

8. An intraluminal stent delivery system comprising:
a catheter; and
a stent disposed on the catheter, the stent comprising a body including at least one therapeutic agent coating positioned on the body and positioned substantially on an interface portion to provide an optimal drug release profile.

9. The system of claim 8 wherein the interface portion comprises an outer surface of the body.

10. The system of claim 8 wherein the at least one therapeutic agent coating comprises at least one drug.

11. The system of claim 10 wherein the at least one drug comprises at least one of an anti-inflammatory agent and an antiproliferative agent.

12. The system of claim 8 wherein the at least one therapeutic agent coating comprises at least one of an immobile polymeric layer coating and a mobile polymeric layer coating.

13. The system of claim 8 wherein the at least one therapeutic agent coating comprises a differential elution kinetic.

14. A method of manufacturing an intraluminal device, the method comprising:
providing a body;
applying at least one therapeutic agent coating positioned on the body and distributing the at least one therapeutic agent coating substantially on an interface portion to provide an optimal drug release profile.

15. The method of claim 14 wherein the body comprises a stent.

16. The method of claim 14 wherein the at least one therapeutic agent is applied to an outer surface of the body.

17. The method of claim 14 wherein the at least one therapeutic agent coating comprises at least one drug.

18. The method of claim 17 wherein the at least one drug comprises at least one of an anti-inflammatory agent and an antiproliferative agent.

19. The method of claim 14 wherein the at least one therapeutic agent coating comprises at least one of an immobile polymeric layer coating and a mobile polymeric layer coating.

20. The method of claim 14 wherein the wherein the at least one therapeutic agent coating comprises a differential elution kinetic.
